(19) United States
(12) Patent Application Publication
(10) Pub. No.: US 2025/0382858 A1
(43) Pub. Date: Dec. 18, 2025

(54) METHODS FOR DETERMINING SAFE DENSITY WINDOWS OF HYDRATE FORMATION CONSIDERING MUD CAKES

(71) Applicant: CHANGZHOU UNIVERSITY, Changzhou (CN)

(72) Inventors: Haiqun CHEN, Changzhou (CN); Haoping PENG, Changzhou (CN); Xinying CUI, Changzhou (CN); Shifeng ZHANG, Changzhou (CN); Dafang HE, Changzhou (CN); Yan ZHUANG, Changzhou (CN); Wenke CAO, Changzhou (CN); Qinze XING, Changzhou (CN); Yuqing XU, Changzhou (CN); Jiabao LI, Changzhou (CN)

(73) Assignee: CHANGZHOU UNIVERSITY, Changzhou (CN)

(21) Appl. No.: 19/238,520

(22) Filed: Jun. 15, 2025

Related U.S. Application Data
(63) Continuation of application No. PCT/CN2025/097710, filed on May 28, 2025.

(30) Foreign Application Priority Data
Jun. 14, 2024 (CN) 202410769873.2

(51) Int. Cl. E21B 41/00; E21B 49/00

(52) U.S. Cl. CPC E21B 41/0099; E21B 49/005; E21B 49/006; E21B 2200/20

(57) ABSTRACT

A method for determining a safe density window of a hydrate formation considering a mud cake under an action of drilling fluids is provided. The method establishes a heat-fluid-solid-chemical multi-field coupling model considering the seepage effect of the mud cake at the well wall and the influence of natural gas hydrate decomposition. The simulation results show the distribution of pore pressure, temperature, and solute concentration in the drilling fluid around the well after the drilling fluid invades. Based on the determination of multi-field coupling model, the determination results, combined with a Cullen-Moore criterion, and manner for calculating a safe density window of a hydrate formation considering the mud cake under the action of the drilling fluid is further established.

### CROSS-REFERENCE TO RELATED APPLICATIONS

This application is a Continuation of International Application No. PCT/CN2025/097710, filed on May 28, 2025, which claims priority to Chinese Patent Application No. 202410769873.2, filed on Jun. 14, 2024, the entire contents of each of which are hereby incorporated by reference.

### TECHNICAL FIELD

The present disclosure relates to the field of petroleum engineering technology, and in particular, to methods for determining safe density windows of hydrate formation considering mud cakes.

### BACKGROUND

Borehole destabilization is a critical issue in the petroleum industry and a main cause of stuck tubing, poor logging quality, and poor cementing operations. Mud leakage due to borehole fracturing is another problem that leads to borehole destabilization. These are common problems in hydrate formation, initially caused by rock stresses exceeding rock strength during drilling. Economic losses due to well instability in drilling operations exceed $1 billion annually, with lost time accounting for over 40% of all drilling-related non-productive time.

After drilling, the original stress field is disturbed, and the rock around the borehole must carry the loads previously supported by the drilled rock. As a result, there is a concentration of stress around the borehole and, depending on the strength of the rock, the concentration of stress can lead to borehole collapse. However, a purely mechanical analysis is insufficient to evaluate borehole instability in the hydrate formation, and understanding the interactions between drilling fluid and the hydrate formation is absolutely essential. There are different chemicals in the drilling fluid that can chemically interact with the hydrate in the hydrate formation and affect the generation or decomposition of the hydrate. The hydrates in the hydrate formation are interconnected by particles or cemented in the pores of the “sediment matrix”, which strengthens the natural gas-containing hydrate sediments. Decomposition of the hydrates by the drilling fluid reduces the mechanical strength of the rock around the borehole.

Therefore, it is desired to provide a method for determining a safe density window of a hydrate formation considering a mud cake under the action of drilling fluid, which accounts for the interaction between the drilling fluid and the hydrate formation to overcome borehole instability.

### SUMMARY

The present disclosure provides a method for determining a safe density window of a hydrate formation considering a mud cake, the method being performed by a processor. The method may include the following operations.

A seepage model of the mud cake and the hydrate formation may be established, a saturation distribution of formation water, a saturation distribution of methane gas, and a saturation distribution of hydrate may be determined based on a finite element software, based on a mass conservation equation of hydrate-bearing formation solute and a mass conservation equation of mud cake solute, a solute transport model of the mud cake and a solute transport model of the hydrate formation may be determined, and a solute solubility distribution may be determined based on the finite element software;

A heat transfer model of the mud cake and a heat transfer model of the hydrate formation may be constructed based on a heat transfer equation of the hydrate formation and a heat transfer equation of the mud cake, and a temperature distribution of a well wall may be determined based on the finite element software.

A skeletal mechanical model of the mud cake and a skeletal mechanical model of the hydrate formation may be constructed, rock deformation around a well may be determined based on the finite element software, a pore pressure distribution at the well wall may be coupled and determined based on an energy field equation, and an effective stress distribution at the well wall may be derived by using a programming software based on the pore pressure distribution at the well wall.

A collapse pressure and a rupture pressure may be determined based on the effective stress distribution according to a Cullen-Moore criterion and a tensile damage criterion, to obtain the safe density window of an interaction between a drilling fluid and the hydrate formation.

A drilling fluid density may be collected by density measuring equipment.

In response to the drilling fluid density not meeting a preset condition, a density adjustment amount may be determined, and the preset condition may be set based on the safe density window.

An operation of a density adjustment apparatus may be controlled based on the density adjustment amount.

The present disclosure also provides a computer device. The computer device may include a memory and a processor. The memory may store a computer program, and the processor may implement the method for determining the safe density window of the hydrate formation considering the mud cake when executing the computer program.

The present disclosure further provides a non-transitory computer-readable storage medium storing a computer program. When a processor executes the computer program, the processor may implement the method for determining the safe density window of the hydrate formation considering the mud cake.

### BRIEF DESCRIPTION OF THE DRAWINGS

In order to more clearly illustrate the technical scheme of the present disclosure, the following will be a brief introduction to the drawings required in the description of the embodiment. Obviously, the illustrations in the following description are only some embodiments of the present disclosure. For a person of ordinary skill in the art, other accompanying drawings can be obtained based on these accompanying drawings without creative labor.

FIG. 1 is a schematic diagram illustrating a process of a method for determining a safe density window of a hydrate formation considering a mud cake according to some embodiments of the present disclosure;

FIG. 2 is a schematic diagram illustrating influence of initial saturation of hydrate on the safe density window according to some embodiments of the present disclosure;

FIG. 3 is a schematic diagram illustrating influence of a permeability of a mud cake on the safe density window according to some embodiments of the present disclosure;

FIG. 4 is a schematic diagram illustrating influence of a temperature of drilling fluid on the safe density window according to some embodiments of the present disclosure;

FIG. 5 is a schematic diagram illustrating influence of a solute concentration on the safe density window according to some embodiments of the present disclosure; and

FIG. 6 is a schematic diagram illustrating a process of another method for determining a safe density window of a hydrate formation considering a mud cake according to some embodiments of the present disclosure.

### DETAILED DESCRIPTION

In order to make the above purposes, features, and advantages of the present disclosure more apparent and understandable, the following is a detailed description of the specific embodiments of the present disclosure in conjunction with the accompanying drawings of the disclosure. It is clear that the described embodiments are a part of the embodiments of the present disclosure, and not all of them. Based on the embodiments in the present disclosure, all other embodiments obtained by a person of ordinary skill in the art without creative labor should fall within the scope of protection of the present disclosure.

Many specific details are set forth in the following description in order to facilitate a full understanding of the present disclosure, but the present disclosure may be carried out in other ways than those described herein. A person skilled in the art may, without departing from the inner meaning of the present disclosure make similar generalizations, and therefore the present disclosure is not limited by the specific embodiments disclosed below.

Second, “an embodiment” or “embodiments” as used herein refers to specific features or characteristics that may be included in at least one embodiment of the present disclosure. The phrase “in one embodiment” as it appears in various places in the present disclosure does not always refer to the same embodiment, nor does it refer to embodiments that are separate or selectively mutually exclusive of other embodiments.

The present disclosure is described in detail in conjunction with the schematic drawings. In the detailed description of the embodiments of the present disclosure, the sectional drawings representing the structure of the device will not be enlarged partially at a general scale for the convenience of illustration, the schematic drawings are only examples, and the sectional drawings shall not limit the scope of protection of the present disclosure herein. In addition, three-dimensional spatial dimensions of length, width, and depth should be included in the actual fabrication.

Meanwhile, in the description of the present disclosure, it is to be noted that the orientation or positional relationships indicated by the terms “upper”, “lower”, “inner”, and “outer” are based on the orientation or positional relationships shown in the accompanying drawings. The terms “up”, “down”, “in”, and “out” indicate orientations or positional relationships based on those shown in the accompanying drawings, are intended only to facilitate and simplify the description of the present disclosure, and are not intended to indicate or imply that the device or element referred to must be constructed and operated in a particular orientation, therefore are not to be construed as a limitation of the present disclosure. Additionally, the terms “first”, “second”, or “third” are used for descriptive purposes only and are not to be construed as indicative of, or suggestive of, relative importance.

In the present disclosure, unless otherwise expressly specified or limited, the terms “mounted”, “attached”, and “connected” are to be broadly construed. For example, a connection may be fixed, removable, or one-piece; likewise, a connection may be mechanical, electrical, direct, indirect through an intermediate medium, or internal to two components. The specific meaning of the above terms in the present disclosure may be understood in specific contexts for those of ordinary skill in the art.

### Embodiment 1

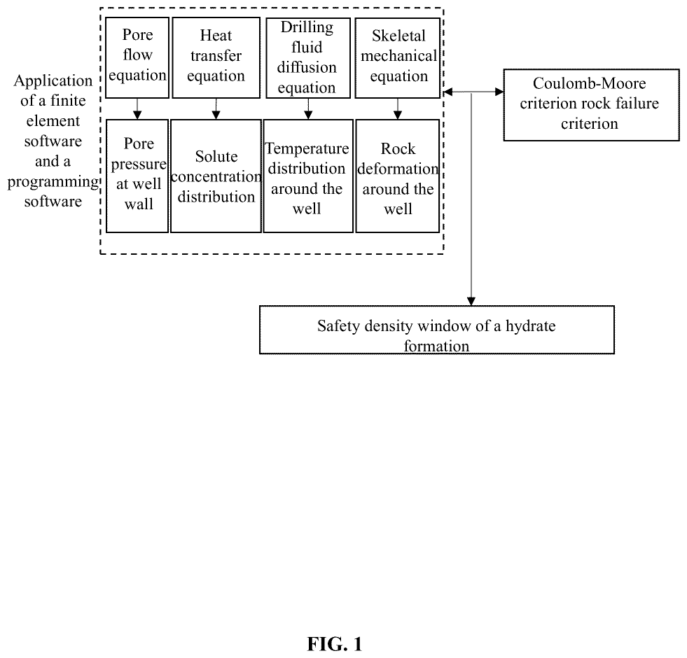

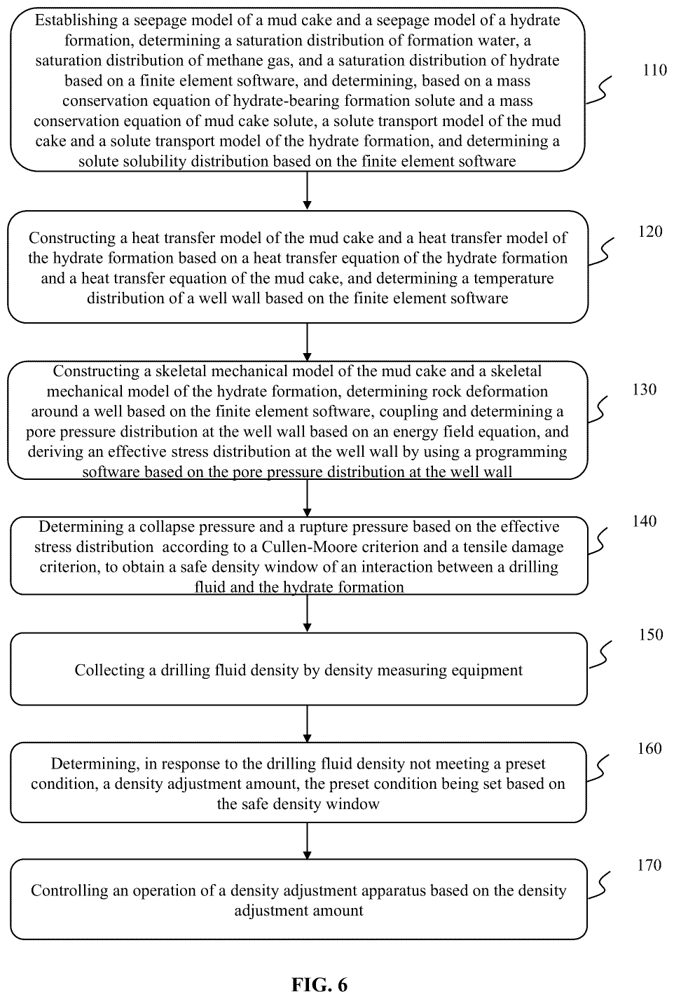

FIG. 1 is a schematic diagram illustrating a process of a method for determining a safe density window of a hydrate formation considering a mud cake according to some embodiments of the present disclosure. FIG. 6 is a schematic diagram illustrating a process of another method for determining a safe density window of a hydrate formation considering a mud cake according to some embodiments of the present disclosure. The method is executed by a processor. The processor may include a central processing unit, a digital signal processor, or the like. The processor may be located on a mobile terminal. The mobile terminal may include a desktop computer, a tablet computer, or the like.

Referring to FIG. 1 and FIG. 6, the embodiment 1 of the present disclosure is shown. The methods shown in FIG. 1 and FIG. 6 may be used to determine the safe density window of the hydrate formation. Descriptions regarding the safe density window of the hydrate formation may be found in the description in embodiment 2. The embodiment provides a method for determining a safe density window of a hydrate formation considering a mud cake, which includes operations 110-170 as follows.

In 110, a seepage model of the mud cake and a seepage model of the hydrate formation is established, a saturation distribution of formation water, a saturation distribution of methane gas, and a saturation distribution of hydrate are determined based on a finite element software, based on a mass conservation equation of hydrate-bearing formation solute and a mass conservation equation of mud cake solute, a solute transport model of the mud cake and a solute transport model of the hydrate formation are determined, and a solute solubility distribution is determined based on the finite element software.

The seepage model may be configured to analyze a pore pressure distribution in the hydrate formation. The seepage model may analyze the pore pressure distribution in the hydrate formation based on the principles of mass conservation and momentum conservation.

In some embodiments, the process of the processor establishing the seepage model of the mud cake and the hydrate formation includes obtaining, from a continuity equation and a generalized Darcy's law, the following seepage information.

A change rate of a mass of the methane gas in the hydrate formation over time is equal to a flow rate of permeability of the hydrate formation under an action of a pressure gradient of the methane gas plus a gas production rate of hydrate dissociation.

A change rate of a mass of water in the hydrate formation over time is equal to a flow rate of permeability of formation under an action of a pressure gradient of the water plus a water production rate of the hydrate dissociation.

A change rate of a mass of the hydrate in the hydrate formation over time is equal to a hydrate rate of the hydrate dissociation.

Assuming that only an aqueous phase exists in pores of the mud cake and that a flow is in accordance with the generalized Darcy's law.

Then a product of a change rate of a pressure of the formation water in the mud cake over time and a ratio of a porosity of the mud cake to a shear modulus of the mud cake is equal to a flow rate of the formation water in the mud cake.

Merely by way of example, the process of the processor establishing the seepage model of the mud cake and the hydrate formation includes obtaining, from the continuity equation and the generalized Darcy's law, the following seepage equation:

$$\begin{matrix}\frac{\partial ( ϕ {ρ}_{g} {S}_{g} )}{\partial t} = ∇ ( ∇ {P}_{g} \frac{{k}_{rg} {ρ}_{g}}{{μ}_{g}} {k}_{r} ) + {\dot{m}}_{g} \\ \frac{\partial ( ϕ {ρ}_{w} {S}_{w} )}{\partial t} = ∇ ( ∇ {P}_{w} \frac{{k}_{rw} {ρ}_{w}}{{μ}_{w}} {k}_{r} ) + {\dot{m}}_{w} \\ \frac{\partial ( ϕ {ρ}_{h} {S}_{h} )}{\partial t} = - {\dot{m}}_{h} .\end{matrix}$$

Assuming that only the aqueous phase exists in the pores of the mud cake and that a flow is in accordance with the generalized Darcy's law, then:

$$\frac{{ϕ}_{s}}{{G}_{s}} \frac{\partial {P}_{w}}{\partial t} = ∇ ( ∇ {P}_{w} \frac{{k}_{s} {ρ}_{w}}{{μ}_{w}} ) .$$

kᵣ and kₛ denote a permeability of the hydrate formation and a permeability of the mud cake; k_{rg} and k_{rw} denote a relative permeability of the methane gas and a relative permeability of water, respectively; μ_{g} and μ_{w} denote the assumed constant viscosity of the methane gas and water; ϕ denotes the porosity; P_{g} and P_{w} denote a pressure of the methane gas and a pressure of water; ρᵣ, ρ_{w}, ρ_{g}, and ρₕ denote mass densities of skeleton, the water, the methane gas, and the hydrate of the hydrate formation, respectively; S_{w}, S_{g}, and Sₕ denote the saturation of water, methane, and the hydrate of the hydrate formation; \dot{m}_{g}, \dot{m}_{w}, and \dot{m}ₕ denote rates of hydrate dissociation, gas production, and water production rate per unit volume; Gₛ denotes the shear modulus, and t denotes the time; and ϕₛ denotes the porosity of the mud cake.

kᵣ, ϕ, ϕₛ, kₛ, k_{rg}, k_{rw}, μ_{g}, μ_{w}, ρᵣ, ρ_{w}, ρ_{g}, ρₕ, \dot{m}_{g}, \dot{m}_{w}, \dot{m}ₙ, and Gₛ may be obtained by experimental measurements. Initial pressures of P_{g} and P_{w} may be obtained from field data or simulated initialization, and the subsequent pressures of P_{g} and P_{w} need to be obtained by solving the seepage equation. S_{w}, S_{g}, and Sₕ may be obtained from field data or simulation initialization, and subsequent saturations need to be obtained by solving the mass conservation equation. t may be obtained by timing tools.

The finite element software may include software such as comsol.

The saturation distributions of the formation water, the methane gas, and the hydrates refer to the variation of the volumetric proportions of the formation water, the methane gas, and the hydrates in the formation pores with spatial location.

In some embodiments, the processor may launch the finite element software, and the finite element software may then output the saturation distributions of the formation water, the methane gas, and the hydrates based on the seepage equation and conditions set forth previously.

In some embodiments, the processor determining the saturation distributions of the formation water, the methane gas, and the hydrate includes determining a decomposition rate of the hydrate using the following manner.

The decomposition rate of the hydrate is determined by a ratio of a contact area of the hydrate contacting a water interface to an Avogadro constant, a ratio of water molecules to gas molecules, a collision cross-sectional area of the water, a dissolution kinetic constant, a collision cross-sectional area of gas, and a desorption kinetic constant.

The desorption kinetic constant is determined by a self-diffusion coefficient of the gas, a proportion of an uncovered surface of the hydrate, gas composition, and a relationship between a fugacity of the gas molecules and an equilibrium fugacity in a liquid phase.

The dissolution kinetic constant is determined by a self-diffusion coefficient of the water molecules, water molecule composition and enthalpies of phase transitions of hydrate lattice, a blocking coefficient of solute diffusion, a length of a core sample, a temperature, an initial temperature, and a gas constant.

A relationship between an output rate of methane gas and the water and the decomposition rate of the hydrate is as follows.

The output rate of methane gas is proportional to the decomposition rate of the hydrate, with a proportionality coefficient being a ratio of a mass fraction of the methane gas to a mass fraction of the hydrate and sign being opposite.

The output rate of the water is proportional to the decomposition rate of the hydrate, with a proportionality coefficient being a ratio of a mass fraction of the water to the mass fraction of the hydrate and sign being opposite.

A relationship between the absolute permeability of the hydrate formation and the saturation of the hydrate is as follows.

The absolute permeability of the hydrate formation is determined by a relationship between an intrinsic permeability of a hydrate-free sediment and the saturation of the hydrate, and a permeability decline index characterizes an extent to which the permeability varies with the hydrate saturation.

A linear relationship between a mechanical strength parameter of a layer containing the hydrate and a saturation degree of the layer containing the hydrate is as follows.

A cohesion of the hydrate formation is determined by a cohesion at the saturation of the hydrate of 0 and a linear relationship between a perturbed cohesion of the hydrate and the saturation of the hydrate, and the perturbed cohesion of the hydrate is a constant.

Merely by way of example, the processor determining that the saturation distributions of the formation water, the methane gas, and the hydrate includes calculating the decomposition rate of the hydrate, \dot{m}ₙ using the following model:

$$\begin{matrix}{\dot{m}}_{h} = \frac{{A}^{I}}{{N}_{A}} {( \hat{n} \cdot \frac{{σ}_{w}}{{K}_{w}^{net}} + \frac{{σ}_{g}}{{K}_{g}^{net}} )}^{- 1} \\ {K}_{g}^{net} = {ξ}_{g} \cdot ( 1 - {θ}^{eq} ) \cdot {x}_{g} \cdot ( \frac{{f}_{g}^{2} - {f}_{g}^{{eq}^{2}}}{{f}_{g} \cdot {f}_{g}^{eq}} ) \\ {K}_{w}^{net} = {ξ}_{w} \cdot {x}_{w} \cdot [ 1 - + exp ( - \frac{Δ {h}^{β - L} \cdot ( T - {T}^{ep} )}{{RTT}^{ep}} ) ] .\end{matrix}$$

The relationship between the output rate of the methane gas and the water and the decomposition rate of the hydrate is:

$$\begin{matrix}{\dot{m}}_{g} = - \frac{{\dot{m}}_{h} {M}_{g}}{{M}_{h}} \\ {\dot{m}}_{w} = - \frac{{\dot{m}}_{h} {M}_{w}}{{M}_{h}} .\end{matrix}$$

The relationship between the absolute permeability of the hydrate formation and the saturation of the hydrate is:

$${k}_{r} = {{k}_{0} ( 1 - {S}_{h} )}^{N} .$$

The linear relationship between the mechanical strength parameter of the layer containing the hydrate and the saturation degree of the layer containing the hydrate is:

$$C = {C}_{0} + {C}_{h} {S}_{h} .$$

C₀ denotes the cohesion of the formation when the saturation of the hydrate is 0, Cₕ denotes a perturbed cohesion of the hydrate, Cₕ=2 MPa, A^{I} denotes the contact area of the hydrate contacting the water interface, N_{A} denotes the Avogadro constant; \hat{n} denotes the ratio of water molecules to gas molecules, σ_{w} denotes the collision cross-sectional area;

$${K}_{w}^{net}$$

denotes the dissolution kinetic constant; σ_{g} denotes the collision cross-sectional area;

$${K}_{g}^{net}$$

denotes the dissolution kinetic constant; τ_{g} denotes the self-diffusion coefficient; θ^{eq} denotes the surface coverage of the hydrate, x_{g} denotes the gas composition; f_{g} denotes the fugacity of gas molecules in the liquid phase;

$${f}_{g}^{eq}$$

denotes the equilibrium fugacity, ξ_{w} denotes the self-diffusion coefficient of the water molecules, x_{w} denotes the water molecule composition; Δh^{B-L} denotes the enthalpies of phase transitions of the hydrate lattice; β denotes the blocking coefficient of solute diffusion; L denotes the length of the core sample; T denotes the temperature; T^{ep} denotes the initial temperature; R denotes the gas constant; M_{g} denotes the mass fraction of the methane gas, Mₕ denotes the mass fraction of the hydrate, M_{w} denotes the mass fraction of the water, n denotes a count of the water molecules in the hydrate, k₀ denotes the intrinsic permeability of the hydrate-free sediment; N denotes the permeability decline index; C denotes the cohesion of the formation;

$${f}_{g}^{e {q}^{2}}$$

denotes the square of the fugacity of the gas molecules in the liquid phase, which is a thermodynamic quantity that represents the effective fugacity tendency or chemical formula of gas molecules in the liquid phase and is used to correct deviations in the actual system from ideal gas behavior.

A^{I}, σ_{w}, θ^{eq}, x_{g}, x_{w}, Δh^{B-L}, β, L, T, T^{ep}, R, M_{g}, Mₕ, M_{w}, n, k₀, N, C, C₀, f_{g},

$${f}_{g}^{eq} ′ {ξ}_{w′} {K}_{w}^{net} , and {K}_{g}^{net}$$

may be obtained by experimental measurements, \hat{n} may be determined by the molecular structure of the hydrate, and the decomposition rate \dot{m}ₕ of the hydrate may be obtained by calculating the aforementioned equation.

The solute transport model is a model used to describe and predict the transport and distribution of solutes (e.g., contaminants, chemicals, etc.) in porous media (e.g., groundwater, rocks, etc.).

In some embodiments of the present disclosure, the process of determining the solute transport model of the mud cake and the solute transport model of the hydrate formation includes a relationship of the mass conservation equation of the hydrate-bearing formation solute. The relationship is as follows.

The change rate of solute concentration in the hydrate formation over time is balanced with diffusion amount of solute in the hydrate formation.

A solute diffusion coefficient in the hydrate formation is determined by a water saturation in the hydrate formation and a solute diffusion coefficient of the hydrate-free sediment.

A relationship of the mass conservation equation of the mud cake solute is as follows.

A change rate of solute concentration over time is balanced with a diffusion amount of solute in the mud cake.

A solute diffusion coefficient in the mud cake is determined by the porosity of the mud cake, a porosity of the hydrate formation, and a solute diffusion coefficient of the hydrate-free sediment.

Merely by way of example, the processor determines the solute transport model of the mud cake and the solute transport model of the hydrate formation includes the mass conservation equation of the hydrate-bearing formation solute, which is as follows:

$$\begin{matrix}ϕ {S}_{w} \frac{\partial {c}_{p}}{\partial t} - {D}_{e} {∇}^{2} {c}_{p} = 0 ; \\ {D}_{e} = {S}_{w} {D}_{e 0} .\end{matrix}$$

The relationship of the mass conservation equation of the mud cake solute is:

$$\begin{matrix}{ϕ}_{s} \frac{\partial {C}_{p}}{\partial t} - {D}_{e s} {∇}^{2} {c}_{p} = 0 \\ {D}_{e s} = \frac{{Φ}_{s}}{Φ} {D}_{e 0} .\end{matrix}$$

cₚ denotes a solution concentration; Dₑ₀ denotes the solute diffusion coefficient of hydrate-bearing sediment without hydrates; Dₑ and Dₑₛ denote the solute diffusion coefficients of the hydrate sediment and the mud cake, respectively; β denotes the blocking coefficient of the solute diffusion; and Φₛ denotes the porosity of the formation skeleton.

cₚ may be obtained by solving the mass conservation equation of the solute, Dₑ and Dₑₛ may be obtained by calculating the relationship between saturation and porosity, Dₑ₀ and β may be obtained experimentally, and Φₛ may be obtained by field measurements.

A solute concentration distribution refers to the variation of concentrations of the solute (e.g., contaminants, chemicals, etc.) at different locations in a spatial region (e.g., the drilling fluid, rocks, etc.).

The finite element software may also include software such as COMSOL Multiphysics, ANSYS Fluent, etc.

In some embodiments, the finite element software may utilize the mass conservation equation of the solute of the hydrate formation and the mass conservation equation of the solute of the mud cake to build a system of finite element equations in the finite element software, and then iteratively solve the discretized system of finite element equations to obtain the solute concentration distribution. The processor may obtain the solute concentration distribution by the finite element software.

In 120, the heat transfer model of the mud cake and the heat transfer model of the hydrate formation are constructed based on the heat transfer equation of the hydrate formation and the heat transfer equation of the mud cake, and the temperature distribution of the well wall is determined based on the finite element software.

The heat transfer model is used to describe a heat transfer process in porous media. The heat transfer process may include heat conduction, heat convection, heat radiation, or the like. The heat transfer model may be used to analyze the temperature distribution and a rate of heat transfer in the downhole porous medium based on the principle of energy conservation.

In some embodiments, the processor constructing the heat transfer model of the mud cake and the heat transfer model of the hydrate formation includes a heat transfer relationship in a water-bearing sediment controlled by heat conduction and heat convection. The heat transfer relationship is as follows.

The heat exchange in the water-bearing sediment is affected by both heat conduction and heat convection, and the heat exchange in the water-bearing sediment is balanced with heat released during a hydrate decomposition process.

An equivalent specific heat of the sediment is determined by a heat capacity of a skeleton of the hydrate formation and a heat capacity of the hydrate, the water, and the methane gas.

A thermal conductivity of the water-bearing sediment is determined by a thermal conductivity of the hydrate formation and a thermal conductivity of the water, the methane gas, and the hydrate.

A heat transfer relationship in the mud cake is as follows.

The variation of the heat energy of the mud cake over time is balanced with an amount of heat conduction.

Merely by way of example, the processor constructing the heat transfer model of the mud cake and the heat transfer model of the hydrate formation includes a heat transfer equation in the water-bearing sediment controlled by heat conduction and heat convection, which is as follows.

$$∇ ( {λ}_{c} ∇ T ) + ∇ ( ( {ρ}_{g} {C}_{g} \frac{{K}_{r} {K}_{r g}}{{μ}_{g}} ∇ {P}_{g} + {ρ}_{w} {C}_{w} \frac{{K}_{r} {K}_{r w}}{{μ}_{w}} ∇ {P}_{w} ) T ) - {\dot{m}}_{h} Δ H = \frac{\partial ( T {C}_{t} )}{\partial t} ;$$

$${C}_{t} = ( 1 - φ ) {ρ}_{r} {C}_{r} + φ ( {S}_{h} {ρ}_{h} {C}_{h} + {S}_{w} {ρ}_{w} {C}_{w} + {S}_{g} {ρ}_{g} {C}_{g} ) ;$$

$${λ}_{c} = ( 1 - φ ) {λ}_{r} + φ ( {S}_{w} {λ}_{w} + {S}_{g} {λ}_{g} + {S}_{h} {λ}_{h} ) .$$

The heat transfer equation in the mud cake is:

$${ρ}_{s} {C}_{s} \frac{\partial T}{\partial t} + ∇ ( - {λ}_{s} ∇ T ) = 0 .$$

λₛ, λᵣ, λ_{w}, λ_{g}, and λₕ denote the thermal conductivities of the mud cake, the hydrate formation, the water, the methane gas, and the hydrate; Cₛ, Cᵣ, C_{g}, C_{w}, and Cₕ denote specific heats of a plugging layer, the hydrate formation, the water, the methane gas, and the hydrate; ΔH denotes the enthalpy of methane hydrate; λc denotes general thermal conductivity of the hydrate-bearing sediment; φ denotes the porosity of the formation, and Cₜ denotes the general specific heat of the hydrate-bearing sediment, which is the heat required to increase the temperature of unit mass by 1° C. Cₜ is usually influenced by the multiphase composition of the hydrate, pore water, sediment skeleton, and gas composition.

ρr, ΔH, Cₛ, Cᵣ, C_{g}, C_{w}, φ, Cₜ, and Cₕ may be obtained experimentally; λ_{c}, λₛ, λᵣ, λ_{w}, λ_{g}, and λₕ may be obtained by calculating the aforementioned equations.

In some embodiments, the processor constructing the heat transfer model of the mud cake and the heat transfer model of hydrate formation further includes obtaining a matrix equilibrium relationship of the hydrate formation based on an effective stress principle and an elastic-plastic mechanics theory, which includes the following operations.

An effective stress gradient of the hydrate formation is balanced with a gradient of a product of a pore pressure and a Biot constant.

A value of a Kronecker preset function is set to 1 when an action direction of an effective stress in the hydrate formation is the same as a plane direction of the action direction applied to the hydrate formation.

A value of the Kronecker preset function is set to 0 when the action direction of the effective stress in the hydrate formation is different from the plane direction of the action direction applied to the hydrate formation.

Merely by way of example, the processor constructing the heat transfer model of the mud cake and a heat transfer model of hydrate formation further includes obtaining a matrix equilibrium equation of the hydrate formation based on the effective stress principle and the elastic-plastic mechanics theory. The matrix equilibrium equation is:

$$\begin{matrix}{σ}_{ij , j} - {( α {δ}_{i j} {P}_{i} )}_{, j} = 0 ; \\ {δ}_{i j} = \{ \begin{matrix}1 ( i = j ) \\ 0 ( i \neq j )\end{matrix} .\end{matrix}$$

σ_{ij,j} denotes an effective stress in the formation; P denotes the pore pressure; α denotes the Biot constant, which is taken as 1 for natural gas formation; δᵢⱼ denotes the Kronecker function; i denotes an action direction of the effective stress in the hydrate formation, and j denotes an action direction of applied in the face direction of the hydrate formation.

P may be obtained from field measurements, α is a preset value and Kronecker function is a preset function, σ_{ij,j} may be obtained by solving the above matrix equilibrium equation. The Kronecker function may be preset empirically for those skilled in the art.

In some embodiments, the processor constructing the heat transfer model of the mud cake and the heat transfer model of the hydrate formation further includes a tensor form of geometric equations, which is as follows.

The strain tensor is determined by an average value of a total displacement u_(i, j) and a total displacement u_(j, i), where u denotes the total displacement, i denotes the action direction of the effective stress of the hydrate formation, and j denotes the plane direction of the action direction applied to the hydrate formation.

Based on elastic-plastic constitutive equations and a Drucker-Prager yield criterion, an incremental form of an elastic constitutive equation indicates as follows.

An effective stress increment is determined by an elastic-plastic matrix tensor and a strain increment.

For straight wells, non-uniform horizontal in situ ground stresses, and taking into account fluid percolation and the pore pressure, a stress state in a well wall envelope is as follows.

A radial stress of the well wall envelope is determined by a product of a wellbore pressure and the pore pressure with the Biot constant.

A tangential stress of the well wall envelope is determined by a maximum horizontal principal stress, a minimum horizontal principal stress, the wellbore pressure, the pore pressure, and an angle.

A pendent stress of the well wall envelope is determined by a vertical stress, the maximum horizontal principal stress, the minimum horizontal principal stress, the wellbore pressure, the pore pressure, and the angle.

Merely by way of example, the processor constructing the heat transfer model of the mud cake and the heat transfer model of hydrate formation further includes the tensor form of geometric equations:

$${ε}_{i j} = ( {u}_{i , j} + {u}_{j , i} ) / 2.$$

εᵢⱼ denotes a strain tensor; u denotes a displacement, i denotes the direction of the effective stress of the hydrate formation, and j denotes the direction of the action direction applied to the hydrate formation.

In some embodiments, based on the elastic-plastic constitutive equations and the Drucker-Prager yield criterion, the incremental form of the elastic constitutive equation is expressed as:

$$d {σ}_{ij} = {D}_{ijk 1} d {ε}_{k 1} ;$$

where dσᵢⱼ denotes effective stress increment; Dᵢⱼₖₗ denotes the elastic-plastic matrix tensor; dεₖ₁ denotes the change in the strain increment; εₖ₁ denotes the strain increment; kl denotes the stiffness matrix index, i denotes the direction of the effective stress of the hydrate formation, and j denotes the plane direction of the action direction applied to the hydrate formation.

For straight wells, non-uniform horizontal in situ ground stresses, and taking into account fluid percolation and the pore pressure, a stress state in a well wall envelope is:

$$\begin{matrix}{σ}_{r} = {P}_{m} - α {P}_{p} ; \\ {σ}_{θ} = {σ}_{H} + {σ}_{h} - 2 ( {σ}_{H} - {σ}_{h} ) cos 2 θ - {P}_{m} - α {P}_{p} ; \\ {σ}_{z} = {σ}_{v} - 2 μ [ ( {σ}_{H} - {σ}_{h} ) ] cos 2 θ - α {P}_{p} .\end{matrix}$$

σᵣ, σ_{θ}, and σ_{z} denote radial, tangential and vertical tangential stresses; σᵥ denotes the vertical stress; σ_{H} denotes the maximum horizontal principal stress; σₕ denotes the minimum horizontal principal stress; Pₘ denotes the wellbore pressure; Pₚ denotes the pore pressure; μ denotes the Poisson's ratio; α denotes the Bivo coefficient; θ denotes the amount of counterclockwise rotation measured counterclockwise from the x-axis until centered on the high side of the wellbore.

σᵣ, σ_{θ}, and σ_{z} may be obtained from field measurements. θ may be determined based on the wellbore geometry and the stress direction. α may be preset empirically for one of the skilled in the art. εₖ₁ and kl may be obtained experimentally.

The temperature distribution at the well wall refers to the variation of temperature at different depths along the well wall.

In some embodiments, the finite element software may use the heat transfer equation to establish the finite element equations in the finite element software and obtain temperature distribution of the well wall by iteratively solving the discretized finite element equations. The processor may obtain the solute concentration distribution through the finite element software.

In 130, a skeletal mechanical model of the mud cake and a skeletal mechanical model of the hydrate formation are constructed, rock deformation around a well is determined based on the finite element software, a pore pressure distribution at the well wall is coupled and determined based on an energy field equation, and an effective stress distribution at the well wall is derived by using a programming software based on the pore pressure distribution at the well wall.

The skeletal mechanical model refers to a model used to describe the mechanical behavior of a solid skeleton in a porous medium. The mechanical behavior may include stress, strain, and deformation. The skeletal mechanical model may analyze, based on the principle of mechanical equilibrium, the response of the solid skeleton downhole under external forces.

In some embodiments, the processor may construct the skeletal mechanical model based on a stress-strain relationship between the mud cake and the hydrate formation.

The rock deformation around the well refers to a change in shape and volume of the rock around the wellbore due to external stresses (e.g., geopathic stresses, fluid pressures, temperature changes, etc.) that occur during the drilling process. The rock deformation around the well may be manifested as compression, expansion, shearing, and fracture of the rock.

A manner for determining the rock deformation around the well is similar to the manner for determining the temperature distribution at the well wall, referring to the descriptions described previously.

The energy field equation refers to a mathematical equation used to describe the distribution and transfer of energy in space. The energy field equation is usually established based on the law of energy conservation and the specific physical processes (e.g., heat conduction, convection, radiation, etc.). The energy field equation may provide reasonable pre-determination for a person skilled in the art based on experience and experimental measurements.

The pore pressure distribution at the well wall refers to the rule of the pore pressure changing with spatial location in the formation surrounding the drilled well. The pore pressure refers to the pressure exerted by formation fluids (e.g., the formation water, the methane gas) in the pores of a rock.

In some embodiments, the processor may solve for the pore pressure distribution at the well wall by the energy field equation, fluid flow equation, and solid mechanics equation corresponding to a coupling relationship. The coupling relationship may include thermal-fluid coupling, fluid-solid coupling, and thermal-solid coupling. The thermal-fluid coupling suggests that changes in temperature affect fluid viscosity and density, thus affecting the fluid flow equation. The fluid-solid coupling suggests that the deformation of the formation changes porosity and permeability, thus affecting the fluid flow equation. The thermal-solid coupling suggests that changes in temperature cause thermal expansion, which affects the stress field and thus the fluid flow equations.

The programming software may include Petrel, Geo Sec, etc.

The effective stress distribution at the well wall refers to a law of the effective stress changing with spatial location in the formation around the well. The effective stress distribution of the well wall may directly affect the deformation, fracture, and stability of the rock.

In some embodiments, the processor may obtain the effective stress distribution at the well wall based on the pore pressure distribution at the well wall by the direct calculation of the programming software.

In 140, a collapse pressure and a rupture pressure are determined based on the effective stress distribution according to a Cullen-Moore criterion and a tensile damage criterion to obtain the safe density window of an interaction between a drilling fluid and the hydrate formation.

The Cullen-Moore criterion refers to a criterion that describes the occurrence of shear damage of a material under shear stress.

The tensile damage criterion refers to a criterion that describes the destruction of a material under tensile stress.

The Cullen-Moore criterion and the tensile damage criterion already exist in the field.

The collapse pressure refers to the minimum pressure at which the rock of the well wall undergoes shear damage due to stress concentration.

The rupture pressure refers to the minimum pressure at which a tensile rupture occurs in the well wall.

The safe density window refers to the range of the drilling fluid density variations allowed during the drilling of the hydrate formations, which can maintain the stability of the well wall and prevent accidents such as collapses, differential pressure jamming, and well leakage. The drilling fluid density refers to mass of the drilling fluid per unit volume, with a unit of g/cm³.

The drilling fluid refers to a fluid that is used to carry rock cuttings, cool a drill bit, and maintain the stability of the well wall during drilling. For example, the drilling fluid may include a water-based drilling fluid, an oil-based drilling fluid, a foam drilling fluid, or the like.

The drilling fluid density has an important effect on well wall pressure control.

The safe density window may be used to guide the drilling fluid density adjustments during drilling to ensure that the drilling operation process is safe, efficient, and economical. The safe density window is a key parameter in drilling engineering.

By setting the drilling fluid density within the safe density window, the well wall is ensured to be stabilized, preventing the well wall from collapsing and avoiding well leakage. By setting the drilling fluid density in the safe density window, the drilling fluid performance may be optimized, resistance during drilling may be reduced, drilling efficiency may be improved, damage to the reservoir may be avoided and the reservoir may be protected. By setting the drilling fluid density within the safe density window, the risk of drilling may also be reduced, accidents such as blowouts, stuck drills, and leakages may be reduced, which ensures safety of drilling personnel. Setting the drilling fluid density in the safe density window also plays a role in environmental protection, which reduces environmental pollution caused by the drilling fluid leakage due to well leakage, etc., and promotes the sustainable development of oil and gas resources.

In some embodiments, the processor may determine the safe density window by model. Specifically, the processor establishes the seepage model of the mud cake and the hydrate formation to solve the saturation distribution of the formation water, the methane gas, and the hydrate; establishes the solute transport model of the mud cake and the hydrate formation to solve the solute concentration distribution based on the mass conservation equation; establishes the heat transfer model of the mud cake and the heat transfer model of the hydrate formation to solve the temperature distribution at the well wall; establishes the skeletal mechanical model of the mud cake and the skeletal mechanical model of the hydrate formation to solve the rock deformation and the pore pressure distribution around the well wall, and then obtain the effective stress distribution at the well wall; determine the collapse pressure and the rupture pressure based on the effective stress at the well wall and the Cullen-Moore criterion and the tensile damage criterion and obtain the safe density window of the interaction between the drilling fluid and the hydrate formation.

In some embodiments, under the action of the drilling fluid, based on a Mohr-Coulomb criterion, the method for determining the safe density window of the hydrate formation considering the mud cake includes the following operations.

When σᵣ serves as a minimum principal stress and σ_{θ} serves as a maximum principal stress, the minimum principal stress σᵣ is determined by the product of the wellbore pressure and the pore pressure with the Biot constant, the maximum principal stress is determined by a product of the maximum horizontal principal stress, the minimum horizontal principal stress, the wellbore pressure and the pore pressure with the Biot constant.

According to the Mohr-Coulomb criterion, when σᵣ serves as the minimum principal stress and σ_{θ} serves as the maximum principal stress, the collapse pressure is determined by the maximum principal stress, the minimum principal stress, an internal friction angle, and a cohesion.

When σᵣ serves as the minimum principal stress and σ_{z} serves as the maximum principal stress, the minimum principal stress σᵣ is determined by the product of the wellbore pressure and the pore pressure with the Biot constant, the maximum principal stress σ_{z} is determined by the vertical stress, a Poisson ratio, the maximum horizontal principal stress, the minimum horizontal principal stress, and the product of the pore pressure and the Biot constant.

According to the Mohr-Coulomb criterion, when σᵣ serves as the minimum principal stress and σ_{z} serves as the maximum principal stress, the collapse pressure is determined by the maximum principal stress, the minimum principal stress, the internal friction angle, and the cohesion.

When σ_{θ} serves as the minimum principal stress and σ_{z} serves as the maximum principal stress, the minimum principal stress σ_{θ} is determined by the product of the maximum horizontal principal stress, the minimum horizontal principal stress, the wellbore pressure, and the pore pressure with the Biot constant, and the maximum principal stress σ_{z} is determined by a product of the vertical stress, the Poisson ratio, the maximum horizontal principal stress, the minimum horizontal principal stress, and the pore pressure with the Biot constant.

According to the Mohr-Coulomb criterion, when σ_{θ} serves as the minimum principal stress and σ_{z} serves as the maximum principal stress, the collapse pressure is determined by the maximum principal stress, the minimum principal stress, the internal friction angle, and the cohesion.

When σ_{θ} serves as the minimum principal stress and σᵣ serves as the maximum principal stress, the minimum principal stress σ_{θ} is determined by the product of the maximum horizontal principal stress, the minimum horizontal principal stress, the wellbore pressure, and the pore pressure, with the Biot constant, and the maximum principal stress σᵣ is determined by the product of the wellbore pressure and the pore pressure with the Biot constant.

According to the Mohr-Coulomb criterion, when σ_{θ} serves as the minimum principal stress and σᵣ serves as the maximum principal stress, the collapse pressure is determined by the maximum principal stress, the minimum principal stress, the internal friction angle, and the cohesion.

When tensile damage occurs in formation, the collapse pressure is determined by the product of the minimum horizontal principal stress, the maximum horizontal principal stress, and the pore pressure with the Biot constant, the tensile strength, and the wellbore pressure.

A manner for determining a density window of safe drilling fluid includes the following operations.

The density window of the safe drilling fluid is determined by the collapse pressure when the tensile damage occurs in the formation and a minimum value of the collapse pressure under different conditions.

Merely by way of example, under an action of the drilling fluids, based on the Mohr-Coulomb criterion, the method for determining the safe density window of the hydrate formation considering the mud cake includes the following operations.

When σᵣ serves as the minimum principal stress and σ_{θ} serves as the maximum principal stress,

$$\begin{matrix}\{ \begin{matrix}{σ}_{3} = {P}_{m} - α {P}_{p} \\ {σ}_{1} = 3 {σ}_{H} - {σ}_{h} - {P}_{m} - α {P}_{p}\end{matrix} ; \\ f ( {P}_{m 1} ) = ( {σ}_{1} - {σ}_{3} ) - ( {σ}_{1} + {σ}_{3} ) sin φ - 2 C cos φ = 0.\end{matrix}$$

When σᵣ serves as the minimum principal stress and σ_{z} serves as the maximum principal stress,

$$\begin{matrix}\{ \begin{matrix}{σ}_{3} = {P}_{m} - α {P}_{p} \\ {σ}_{1} = {σ}_{v} - μ [ 2 ( {σ}_{H} - {σ}_{h} ) ] - α {P}_{p}\end{matrix} ; \\ f ( {P}_{m 2} ) = ( {σ}_{1} - {σ}_{3} ) - ( {σ}_{1} + {σ}_{3} ) sin φ - 2 C cos φ = 0.\end{matrix}$$

When σ_{θ} serves as the minimum principal stress and σ_{z} serves as the maximum principal stress,

$$\begin{matrix}\{ \begin{matrix}{σ}_{3} = 3 {σ}_{H} - {σ}_{h} - {P}_{m} - α {P}_{p} \\ {σ}_{1} = {σ}_{v} - μ [ 2 ( {σ}_{H} - {σ}_{h} ) ] - α {P}_{p}\end{matrix} ; \\ f ( {P}_{m 3} ) = ( {σ}_{1} - {σ}_{3} ) - ( {σ}_{1} + {σ}_{3} ) sin φ - 2 C cos φ = 0.\end{matrix}$$

When σ_{θ} serving as the minimum principal stress and σᵣ serving as the maximum principal stress,

$$\begin{matrix}\{ \begin{matrix}{σ}_{3} = 3 {σ}_{H} - {σ}_{h} - {P}_{m} - α {P}_{p} \\ {σ}_{1} = {P}_{m} - α {P}_{p}\end{matrix} ; \\ f ( {P}_{m 4} ) = ( {σ}_{1} - {σ}_{3} ) - ( {σ}_{1} + {σ}_{3} ) sin φ - 2 C cos φ = 0.\end{matrix}$$

When tensile damage occurs in the formation,

$$f ( {P}_{m 5} ) = 3 {σ}_{h} - {σ}_{H} - α {P}_{p} + {σ}_{t} - {P}_{m} .$$

The manner for determining the density window of safe drilling fluid includes:

$${P}_{m 5} \leq {ρ}_{m} \leq min \{ {P}_{m 1} , {P}_{m 2} , {P}_{m 3} , {P}_{m 4} \} .$$

ρₘ denotes the drilling fluid density; Pₘ₁-Pₘ₅ denotes the collapse pressure under different conditions; μ denotes the viscosity; σ₁ denotes the maximum principal stress; σ₃ denotes the minimum principal stress; σₜ denotes the tensile strength; Pₘ₁ denotes the collapse pressure when σᵣ is used as the minimum principal stress and σ_{θ} is used as the maximum principal stress; Pₘ₂ denotes the collapse pressure when σᵣ is used as the minimum principal stress and σ_{z} is used as the maximum principal stress; Pₘ₃ denotes the collapse pressure when σ_{θ} is used as the minimum principal stress and σ_{z} is used as the maximum principal stress; Pₘ₄ denotes the collapse pressure when σ_{θ} is used as the minimum principal stress and σᵣ is used as the maximum principal stress; and Pₘ₅ denotes the collapse pressure when the formation undergoes tensile failure.

Pₘ₁-Pₘ₅ is obtained by calculation through the Mohr-Coulomb criterion and tensile failure conditions. The maximum principal stress and the minimum principal stress may be obtained by field measurements. The viscosity and the tensile strength may be obtained by experimental measurement.

In some embodiments, the processor may use the drilling fluid density corresponding to the collapse pressure as a lower limit of the safe density window, and the drilling fluid density corresponding to the rupture pressure as an upper limit of the safe density window.

The upper limit of the safe density window refers to a critical density at which the drilling fluid density exceeds the value that could lead to the well wall rupture.

The lower limit of the safe density window refers to a critical density below which the drilling fluid density may trigger the well wall collapse.

In some embodiments, the processor may determine the drilling fluid density corresponding to the collapse pressure using the following first algorithm.

The drilling fluid density corresponding to the collapse pressure=the collapse pressure P/(gravitational acceleration g*well depth H).

In some embodiments, the processor may determine the drilling fluid density corresponding to the rupture pressure using the following second algorithm.

The drilling fluid density corresponding to the rupture pressure=the rupture pressure P/(the gravitational acceleration g*the well depth H).

In 150, a drilling fluid density is collected by density measuring equipment.

The density measuring equipment refers to equipment used to measure the drilling fluid density. For example, the density measuring equipment includes a densitometer, a funnel viscometer, a density sensor, or the like.

In 160, in response to the drilling fluid density not meeting a preset condition, a density adjustment amount is determined.

The preset condition refers to a condition used to determine whether the drilling fluid density needs to be adjusted.

In some embodiments, the processor may set the preset condition based on the safe density window. For example, the preset condition may be that the drilling fluid density lies in the safe density window.

The density adjustment amount refers to a change value of the density required to make the drilling fluid density meet the preset condition when the drilling fluid density does not meet the preset condition.

In some embodiments, the processor may use a difference between a target drilling fluid density and a current drilling fluid density as the density adjustment amount. In some embodiments, the processor may obtain the current drilling fluid density via the density measuring equipment.

The target drilling fluid density refers to a desired drilling fluid density.

In some embodiments, in response to the drilling fluid density not meeting the preset condition, the processor may select the target drilling fluid density from the safe density window. For example, the processor may randomly select a drilling fluid density from the safe density window as the target drilling fluid density. In some embodiments, in response to the drilling fluid density not meeting the preset condition, the processor may determine the target drilling fluid density based on the upper limit and the lower limit of the safe density window.

In some embodiments of the present disclosure, the processor may take an average of the upper limit and the lower limit of the safe density window, as the target drilling fluid density.

In 170, an operation of a density adjustment apparatus is controlled based on the density adjustment amount.

The density adjustment apparatus refers to an apparatus for adjusting the drilling fluid density of the drilling fluid. The density adjustment apparatus may adjust the drilling fluid density by removing a solid phase from the drilling fluid, adding a weighting material to the drilling fluid, removing the gas from the drilling fluid, and monitoring the drilling fluid density in real-time. For example, the density adjustment apparatus may include solid control equipment, weighting equipment, gas removal equipment, density measuring equipment, other auxiliary equipment, or the like.

The solid control equipment may include a vibrating screen, a grit remover, a mud remover, a centrifuge, etc. The vibrating screen may be configured to remove large particles of the solid phase in the drilling fluid and reduce impurities, such as drill cuttings, to reduce the drilling fluid density. The mesh size of the screen cloth of the vibrating screen may be adjusted according to the requirement of the drilling fluid density.

The weighting equipment may include a weighting material adding apparatus, a weighted pump, or the like. The weighting material addition apparatus may be configured to uniformly add weighting materials (e.g., barite, iron ore powder, limestone powder, etc.) to the drilling fluid to increase the drilling fluid density.

The gas removal equipment may include a degasser, a liquid-gas separator, or the like.

The density measuring equipment may include a funnel viscometer, a densitometer, or the like.

The other auxiliary equipment may include a stirrer, heating equipment, or the like.

In some embodiments of the present disclosure, in response to the density adjustment amount being positive, the processor may, based on the density adjustment amount, weight the drilling fluid via the density adjustment apparatus to increase the current drilling fluid density to the target drilling fluid density. A manner for weighting the drilling fluid may include adding the weighting material to the drilling fluid, mixing a high-density drilling fluid into the drilling fluid, optimizing the drilling fluid system, etc.

The operation of adding the weighting material to the drilling fluid may include the processor first removing poor quality solid phase from the drilling fluid by the solid control equipment (e.g., vibrating screen, centrifuge), then adding the weighting material evenly to the drilling fluid and stirring evenly to ensure that the weighting material is fully dispersed.

The operation of mixing the high-density drilling fluid into the drilling fluid may include the processor mixing the prepared high-density drilling fluid into the current drilling fluid in proportion and gradually increasing the overall density of the drilling fluid until the target drilling fluid density is reached. The processor may determine, based on a first preset control table, a proportion of the high-density drilling fluid mixed into the current drilling fluid. The correspondence between the density adjustment amount, the current drilling fluid density, the target drilling fluid density, and the proportion of high-density drilling fluid to be added exists in the first preset control table. The first preset control table may be constructed based on historical data.

The operation of optimizing the drilling fluid system may include the processor adjusting the formulation and performance of the drilling fluid until the target drilling fluid density is achieved based on formation conditions and drilling requirements. For example, the processor may increase bentonite content in the drilling fluid to increase the suspension and sand-carrying capacity of the drilling fluid, thereby indirectly increasing the drilling fluid density.

In some embodiments of the present disclosure, in response to the density adjustment amount being negative, the processor may dilute the drilling fluid based on the density adjustment amount via the density adjustment apparatus to reduce the current drilling fluid density to the target drilling fluid density. A manner for diluting the drilling fluid may include diluting the drilling fluid using the solid control equipment, mixing a low-density fluid in the drilling fluid, or the like.

The operation of diluting the drilling fluid using the solid control equipment may include utilizing the vibrating screen to remove large particles of solid phase from the drilling fluid, reducing the solid phase content, thereby reducing the density. Diluting the drilling fluid using the solid control equipment may also include utilizing the grit remover and the mud remover to further remove fine particles and purify the drilling fluid. Diluting the drilling fluid using the solid control equipment may further include utilizing the centrifuge to separate the solid phase and the liquid phase of the drilling fluid, reducing the amount of the weighting material, and lowering the drilling fluid density.

The operation of mixing the low-density liquid in the drilling fluid refers to an operation of adding a corresponding amount of water to the drilling fluid based on the density adjustment amount to dilute the drilling fluid and reduce the drilling fluid density until the target drilling fluid density is reached. However, it is necessary to pay attention to controlling the amount of the water added, to avoid the drilling fluid density decreasing excessively quickly. The processor may determine the corresponding amount of clear water based on the density adjustment amount by a second preset control table. In the second preset control table, there exists a correspondence between the current drilling fluid density, the target drilling fluid density, the density adjustment amount, and the amount of clear water. The second preset control table may be preset for a person skilled in the art based on experience.

In some embodiments of the present disclosure, in response to the current drilling fluid density being increased to the target drilling fluid density, the processor may also perform a gas removal operation or a drilling fluid formulation adjustment operation on the drilling fluid.

The gas removal operation may include removing gas (e.g., natural gas, carbon dioxide) from the drilling fluid by the degasser to prevent gas intrusion from causing unanticipated reductions in density and affecting rock wall stability, or separating the gas and liquid by the liquid-gas separator to ensure that the drilling fluid density is stabilized.

The drilling fluid formulation adjustment operation refers to the improvement of reducing the amount of the weighting material, optimizing the formulation and performance of the drilling fluid, and making the drilling fluid more suitable for the current drilling condition.

In some embodiments of the present disclosure, the method for determining the safe density window of the hydrate formation considering the mud cake may further include operations 180-200 as follows.

In 180, underground environmental data is collected through monitoring equipment.

The monitoring equipment refers to equipment configured to collect data of the underground environment. For example, the monitoring device includes at least one of a temperature sensor, a humidity sensor, a gas sensor, a pressure sensor, or the like. The acquisition frequency of each sensor may be preset for a person skilled in the art based on experience.

The underground environmental data refers to data related to the downhole environment. For example, the underground environmental data may include at least one of ambient temperature, humidity, gas composition, or the like for a preset historical time period downhole. The preset historical time period may be preset empirically for a person skilled in the art.

In 190, a correction window is determined by a correction model based on the safe density window, pressure data and density data of the drilling fluid, a chemical composition concentration of the drilling fluid, and the underground environmental data.

The pressure data and the density data of the drilling fluid are pressure data and density data of the drilling fluid in the wellbore for a preset historical time period.

In some embodiments, the processor may obtain the pressure data of the drilling fluid via a pressure sensor and the density data of the drilling fluid via a density sensor.

The chemical composition concentration of the drilling fluid refers to the concentration of each chemical component (e.g., an inhibitor, a weighting material, a filtration loss reducer, etc.) in the drilling fluid over a preset historical time period.

In some embodiments, the processor may obtain the chemical composition concentration of the drilling fluid via a laboratory instrument.

The correction window refers to a safe density window after correction.

In some embodiments, the correction model is a machine learning model. In some embodiments, the correction model may be any one or a combination of a neural network (NN) model, a deep neural network (DNN) model, etc., or other customized model structures, etc.

In some embodiments, the correction model may be obtained by training based on a plurality of first training samples with a first label.

In some embodiments, each first training sample may include a historical sample safe density window, historical sample pressure data, historical sample density data, historical sample chemical composition concentration, and historical sample underground environmental data corresponding to the same historical sample of the drilling fluid.

In some embodiments, the first label may be an actual safe density window corresponding to the first training sample. The actual safe density window refers to a safe density window actually used in drilling operations after experts perform field corrections. The field corrections may be based on manual adjustment of the safe density window by the experts based on specific characteristics of the hydrate formation at the drilling site (e.g., rock strength, fracture development, etc.).

The first training samples and the first label may be obtained based on historical data. The historical data may include data from a plurality of drilling logs performed during a preset historical time period. Each of the plurality of pieces of drilling log data includes a historical safe density window, historical pressure data, historical density data, historical chemical composition concentration, historical underground environmental data, and an actual safe density window corresponding to the same historical drilling fluid in the preset historical time period. The preset historical time period may be divided into a plurality of historical sub-preset historical time periods. The preset historical time period, the manner for dividing into historical sub-preset time periods, and the historical sub-preset historical time periods may all be preset by experience for those skilled in the art.

In some embodiments, the processor may determine a plurality of first preferred cases from the historical data, generating a first training sample and a corresponding first label based on each first preferred case.

The first preferred case refers to drilling log data when the drilled well has not collapsed or leaked in the historical data.

In some embodiments, the processor may perform a plurality of rounds of iterations. When an end-of-iteration condition is satisfied, the iteration is ended to obtain a trained and completed correction model. The end-of-iteration condition may include a count of iterations reaching a count threshold, etc.

In some embodiments, performing a round of iteration by the processor may include selecting a first training sample from the plurality of first training samples; inputting the first training sample into an initial correction model to obtain a predicted output of the initial correction model corresponding to the first training sample; according to the predicted output, and the first label corresponding to the first training sample, substituting the predicted output and the first label into a predefined loss function to calculate a value of the loss function; and according to the value of the loss function, inversely updating model parameters in the initial correction model.

In some embodiments, the processor may use various algorithms to reverse update model parameters in the initial correction model. For example, the processor may reverse update model parameters in the initial correction model based on a gradient descent algorithm.

In 200, the safe density window is corrected based on the correction window.

In some embodiments, the processor may correct the safe density window as the correction window.

In some embodiments of the present disclosure, correction of the safe density window by the correction model may further improve the accuracy of the finalized safe density window and reduce the probability of occurrence of borehole destabilization during drilling of the hydrate formation.

In some embodiments, the method for determining the safe density window of the hydrate formation considering the mud cake may further include the following operation 210.

In 210, initial conditions of the seepage model, the heat transfer model, and the skeletal mechanical model are updated based on the underground environmental data.

The initial conditions for the seepage model refer to initial pressure preset values of the methane gas and the water in the seepage model. The initial pressure preset value of the methane gas refers to a pressure preset value of the methane gas used for the first time in the seepage model. The initial pressure preset value of the water refers to a pressure preset value of the water used for the first time in the seepage model.

The initial conditions of the heat transfer model refer to the initial temperature field of the heat transfer model. The initial temperature field consists of the temperature distribution at the well wall of each region downhole. The initial temperature field refers to a temperature field first used in the transfer model.

The initial conditions of the skeletal mechanical model refer to the initial pore pressure distribution at the well wall of the skeletal mechanical model. The initial pore pressure distribution at the well wall refers to the pore pressure distribution at the well wall first used in the skeletal mechanical model.

In some embodiments, the processor may update the pressure preset values of the methane gas and water in the model by downhole real-time pressure data obtained through the pressure sensor.

In some embodiments, the processor may update the initial temperature field of the heat transfer model through downhole real-time temperature data collected by the temperature sensor to ensure that the heat transfer model reflects the current downhole field ambient temperature.

In some embodiments, the processor may update the initial pore pressure distribution at the well wall of the skeletal mechanical model through real-time downhole pressure data collected by the pressure sensor.

In some embodiments of the present disclosure, the accuracy of the safe density window of the drilling fluid may be further improved by updating the initial conditions of the seepage model, the heat transfer model, and the skeletal mechanical model based on the underground environmental data.

In some embodiments, the method for determining the safe density window of the hydrate formation considering the mud cake may further include operations 220-230 as follows.

In 220, a plurality of sets of candidate adjustment parameters are generated based on the drilling fluid density and the target drilling fluid density by a parameter determination model.

The candidate adjustment parameters refer to adjustment parameters to be determined. The adjustment parameters may include a raw material input amount and equipment operating parameters. The raw material input amount may include input amounts of the inhibitor, the weighting material, the filtration loss reducer, or the like. The equipment operating parameters may include an operating frequency of the solid control equipment, a rotational speed of the stirrer, or the like.

In some embodiments, the parameter determination model is a machine learning model. In some embodiments, the parameter determination model may be any one or a combination of an NN model, a DNN model, or other customized model structures, etc.

In some embodiments, the parameter determination model may be trained based on a plurality of second training samples with second labels.

In some embodiments, each second training sample may include a historical sample drilling fluid density and a historical sample target drilling fluid density corresponding to the same historical sample drilling fluid. The second labels are a plurality of actual adjustment parameters corresponding to the second training samples.

The second training samples and the second labels may be obtained based on historical data. The historical data may include historical drilling fluid densities and historical target drilling fluid densities corresponding to a plurality of drilling fluid density adjustments made at a historical time, as well as corresponding actual adjustment parameters.

In some embodiments, the processor may determine a plurality of second preferred cases from the historical data and generate a second training sample and a corresponding second label based on each second preferred case.

The second preferred cases refer to records in which the absolute value of the difference between a historical drilling fluid density and a historical target drilling fluid density obtained using historical adjustment parameters (e.g., the raw material input amount and equipment operating parameters) satisfies a preset condition in the historical data. The preset condition may be that the absolute value of the difference between the historical drilling fluid density and the historical target drilling fluid density is less than a preset threshold. The preset threshold may be preset empirically by a person skilled in the art.

In some embodiments, the training process of the parameter determination model is similar to the training process of the correction model, and the detailed descriptions regarding the training process of the parameter determination model may be found in the training process of the correction model hereinabove.

In 230, an optimal adjustment parameter is determined based on the plurality of sets of candidate adjustment parameters.

The optimal adjustment parameter refers to a candidate adjustment parameter that has the best drilling fluid density adjustment effect.

In some embodiments, the processor may evaluate, based on an optimization algorithm, drilling fluid density adjustment effects corresponding to the plurality of sets of the candidate parameters, and determine a candidate adjustment parameter that has the best drilling fluid density adjustment effect from the plurality of sets of candidate adjustment parameters to be the optimal adjustment parameter.

The drilling fluid density adjustment effect refers to the effect of the drilling fluid density adjustment. In some embodiments, based on the candidate adjustment parameters, the smaller the absolute value of the difference between the adjusted actual drilling fluid density and the desired drilling fluid density, the better the effect of the drilling fluid density adjustment corresponding to the candidate adjustment parameter.

In some embodiments, the optimization algorithm may include a genetic algorithm, or the like.

The optimization process of the genetic algorithm is as follows. A plurality of sets of candidate adjustment parameters generated by the neural network are used as a population. Crossover, mutation, and selection operations are performed on the population based on an evaluation function (also known as a fitness function) to retain and pass the candidate adjustment parameters with high fitness (the fitness value is determined by the effect of the drilling fluid density adjustment), ultimately obtaining the optimal (the best effect of the drilling fluid density adjustment) candidate adjustment parameter.

The evaluation function refers to a function that is used to evaluate the effect of the drilling fluid density adjustment of the candidate adjustment parameter to reflect a relationship between the candidate adjustment parameters and the corresponding effect of the drilling fluid density adjustment.

In some embodiments, the processor may construct the evaluation function using the following manner.

In the simulation environment, the processor may conduct test experiments on the effects of the drilling fluid density adjustments of different combinations of candidate adjustment parameters, record experimental data of a plurality of tests, and analyze the experimental data using the statistical manner (e.g., regression analysis) to establish the evaluation function of the effect of the drilling fluid density adjustment.

The experimental data may include the drilling fluid density, the target drilling fluid density, the candidate adjustment parameters, and the absolute value of the difference between the adjusted actual drilling fluid density and the target drilling fluid density (reflecting the effect of drilling fluid density adjustment).

In some embodiments of the present disclosure, when the drilling fluid density needs to be adjusted, the optimal adjustment parameter (e.g., the raw material input amount and equipment operating parameters) is determined by the plurality of sets of the candidate adjustment parameters determined based on the parameter determination model, and the optimization algorithm to improve the accuracy of determining the adjustment parameters, thereby reducing the wasted raw materials and time caused by adjusting the adjustment parameters back and forth.

The present disclosure establishes the physical model of the wellbore, the seepage model of the mud cake and the seepage model of the hydrate formation, the solute transport model of the mud cake and the solute transport model of the hydrate formation, the heat transfer model of the mud cake and the heat transfer model of the hydrate formation, and the skeletal mechanical model of the mud cake and the skeletal mechanical model of the hydrate formation, with simulation results presenting the pore pressure distribution, the temperature distribution, the solute concentration distribution, the water-gas-hydrate saturation distribution, and the effective stress distribution around the formation well after the drilling fluid intrusion. After understanding the formation conditions, according to the Cullen-Moore criterion, the method for determining the safe density window of the hydrate formation under the action of the drilling fluid is provided, which provides a basis for the design of anti-collapse performance of drilling fluid in the drilling process of the hydrate formation.

The method comprehensively considers various factors that affect the stability of the well wall during hydrate extraction, allowing model results to more accurately reflect the actual situation of the formation, and provide a basis for the design of the anti-collapse performance of the drilling fluid in the drilling process of the hydrate formation.

### Embodiment 2

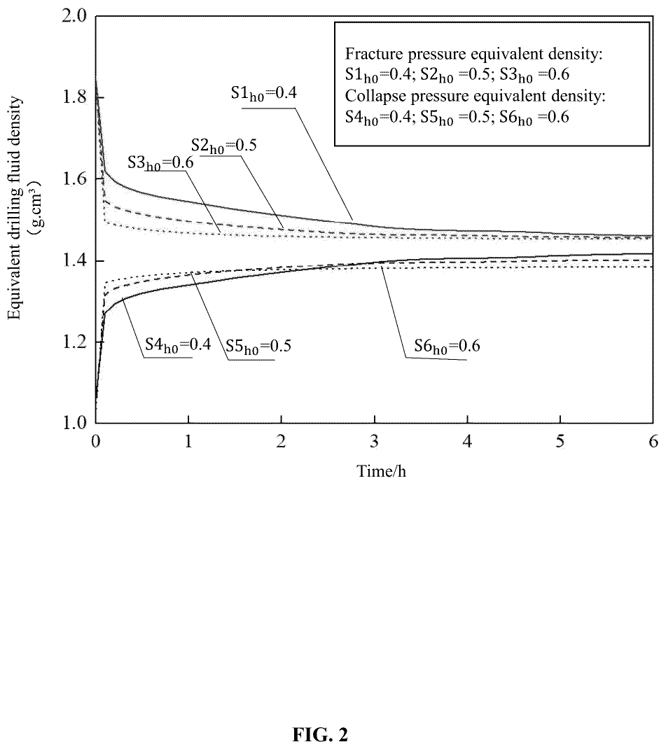

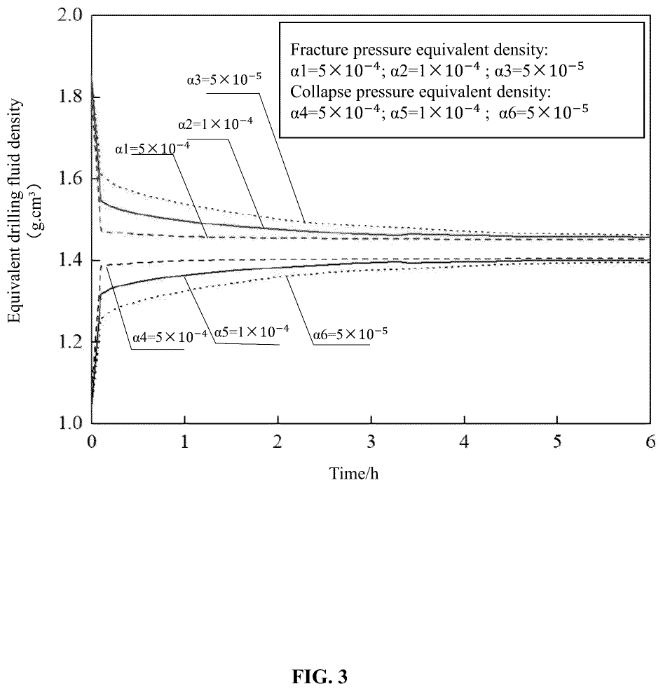

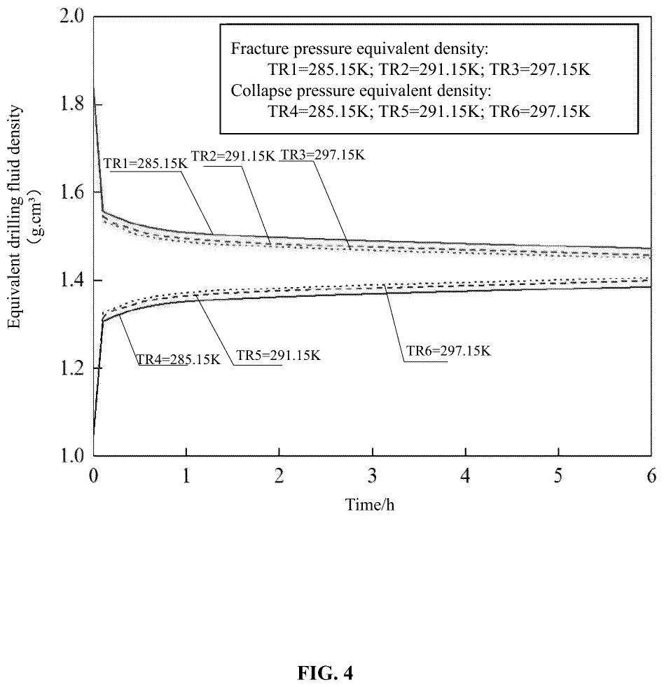

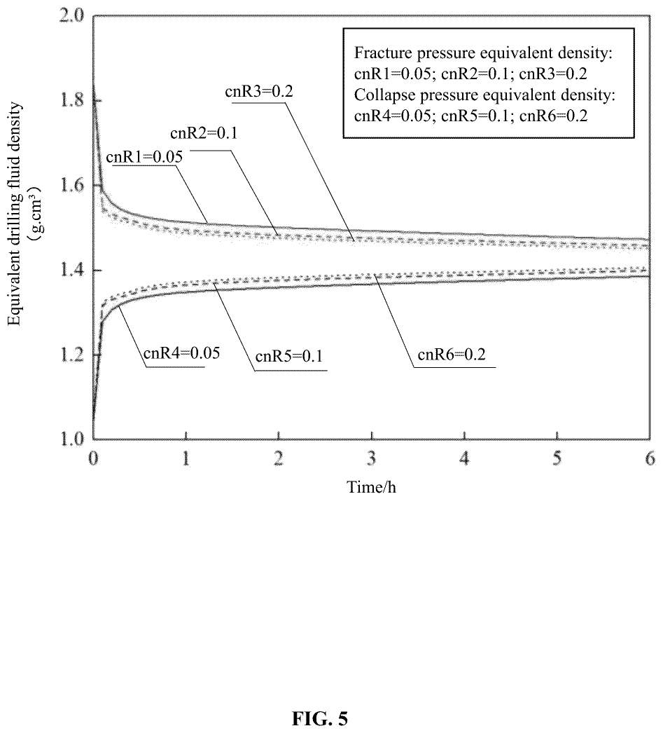

FIG. 2 is a schematic diagram illustrating influence of initial saturation of hydrate on the safe density window according to some embodiments of the present disclosure. FIG. 3 is a schematic diagram illustrating influence of a permeability of a mud cake on the safe density window according to some embodiments of the present disclosure. FIG. 4 is a schematic diagram illustrating influence of a temperature of drilling fluid on the safe density window according to some embodiments of the present disclosure. FIG. 5 is a schematic diagram illustrating influence of a solute concentration on the safe density window according to some embodiments of the present disclosure. Referring to FIG. 2-FIG. 5, an embodiment of the present disclosure provides a method for determining a safe density window of a hydrate formation considering a mud cake. In order to verify the beneficial effect of the present disclosure, it is scientifically demonstrated by experimentation.

The geological system modeled in the present disclosure is located in the South China Sea. The borehole radius (r) was set to 0.5 m, the mud cake thickness (d) was set to 1 mm, and ¼ circle was taken as the study region. The computational parameters of the model, the fixed-solution boundary conditions, and initial values are shown in Tables 1 and 2.

TABLE 1
Parameters related to the hydrate formation in a target block.

| Parameter | Value | | Parameter | Value | |
|---|---|---|---|---|---|
| Vertical stress σᵥ | 21.8 | MPa | Initial hydrate saturation Sₕ₀ | 0.5 | |
| Maximum horizontal principal stress σ_{H} | 20.45 | MPa | Initial hydrate saturation S_{w0} | 0.4 | |
| Minimum horizontal principal stress σₕ | 19.7 | MPa | Initial porosity of formation ϕ₀ | 0.4 | |
| Initial cohesion of formation C₀ | 1 | MPa | Initial permeability of formation k₀ | 0.1 | μm² |
| Tensile strength of formation σₜ | 2 | MPa | Formation initial temperature T₀ | 12.2° | C. |
| Internal friction angle | 25° | | Initial pressure of formation P₀ | 14.434 | MPa |
| Poisson's ratio v | 0.43 | | Initial NaCl concentration cNaCl₀ | 3.05% | wt |
| Methane density ρ_{g} | 0.77 | kg/m³ | Specific heat of methane C_{g} | 2.93 | |
| Water density ρ_{w} | 1030 | kg/m³ | Specific heat of water C_{w} | 0.4 | |
| Hydrate density ρₕ | 910 | kg/m³ | Hydrate specific heat Cₕ | 0.4 | |
| Rock density ρᵣ | 2600 | kg/m³ | Specific heat of rock Cᵣ | 0.1 | μm² |
| Gas viscosity μ_{g} | 2.45e−6 | Pa · s | Gas thermal conductivity λ_{g} | 0.07 | W/(m · K) |
| Water viscosity μ_{w} | 1e−3 | Pa · s | Hydrate thermal conductivity λₕ | 0.53 | W/(m · K) |
| Water depth | 1310 | m | Rock thermal conductivity λᵣ | 3.1 | W/(m · K) |
| Enthalpy of methane hydrate ΔH | 54.2 | kJ/mol | Thermal conductivity of hydrate λ_{w} | 0.6 | W/(m · K) |

TABLE 2
Initial values and boundary conditions

| Physical field | Initial value (t = 0) | Boundary condition |
|---|---|---|
| Seepage field | P_{w} = P₀ | P_{w} (x = R) = 2 × 107 Pa |
| | S_{w} = S_{w0} | P_{w} ( x = ∞) = P₀ |
| | Sₕ = Sₕ₀ | ${S}_{w} ( x = R + d ) = \frac{- {K}_{s} {ρ}_{w}}{{μ}_{w}} \frac{{dP}_{w}}{dr}$ |
| | | S_{w} (x = ∞) = S_{w0} |
| | | cNaCl(x = R) = cnR (take 0.1) |
| Solute motion field | cNaCl = cNaCl0 | |
| | | cNaCl (x = ∞) = cNaCl0 |
| | | T(x = R) = TR (take 291.25) |
| Temperature field | T = T₀ | |
| | | T (x = ∞) = T₀ |
| | | σ (x = R = αPₚ ) |
| Mechanical field | | σ(x = ∞) = original ground stress |

The present disclosure calculates the safe density window of the well wall in the hydrate formation according to the Cullen-Moore criterion using finite element numerical simulation software in conjunction with the programming software to analyze the drilling fluid temperature, the drilling fluid plugging performance, the mud cake thickness, the mud cake permeability, the effects of the mud cake thickness and the hydrate saturation on the pore pressure, the temperature distribution, the solute transport, and the hydrate decomposition rate of the well wall, and further analyze the effects of the drilling fluid on the safe density window of the well wall. The horizontal coordinates of FIG. 2-FIG. 5 all represent time, and the vertical coordinates represent the equivalent drilling fluid density. Based on FIG. 2-FIG. 5, the conclusions are mainly as follows.

First, the six curves in FIG. 2 are curves of equivalent drilling fluid density changing over time when the fracture pressure equivalent density is S1ₕ₀=0.4, S2ₕ₀=0.5, and S3ₕ₀=0.6, and when the collapse pressure equivalent density is S4ₕ₀=0.4, S5ₕ₀=0.5, and S6ₕ₀=0. It can be seen from FIG. 2 that the range of safe density windows for mud in the formation with high hydrate saturation may be greater than in the formation with low hydrate saturation.

Second, the six curves in FIG. 3 are curves of equivalent drilling fluid density changing over time when the fracture pressure equivalent density is α1=5×10⁻⁴, α2=1×10⁻⁴, and α3=5×10⁻⁵, and when the collapse pressure equivalent density is α4=5×10⁻⁴, α5=1×10⁻⁴, and α6=5×10⁻⁵. It can be seen from FIG. 3 that the reduction of the mud cake permeability may widen the safe density window of the drilling fluid in the hydrate formation under the same conditions. The blocking coefficient is reduced from 5×10⁻⁴ to 5×10⁻⁵, and the safe density window is widened from a range of 1.40 g/cm³-1.45 g/cm³ to a range of 1.38 g/cm³-1.48 g/cm³ after 6 hours.

Third, the six curves in FIG. 4 are curves of equivalent drilling fluid density changing over time when the fracture pressure equivalent density is TR1=285.15K, TR2=291.15K, and TR3=297.15K, and when the collapse pressure equivalent density is TR4=285.15K, TR5=291.15K, and TR6=297.15K. It can be seen from FIG. 4 that the drilling fluid with lower temperatures may widen the safe density window of the drilling fluid in the hydrate formation under the same conditions. The drilling fluid temperature increases from 285.15 K to 297.15 K, and the safe density window decreases from a range of 1.38 g/cm³-1.47 g/cm³ to a range of 1.41 g/cm³-1.45 g/cm³ after 6 hours.

Fourth, the six curves in FIG. 5 are curves of equivalent drilling fluid density changing over time when the fracture pressure equivalent density is cnR1=0.05, cnR2=0.1, and cnR3=0.2, and when the collapse pressure equivalent density is cnR4=0.05, cnR5=0.1, and cnR6=0.2. It can be seen from FIG. 5 that the drilling fluid with lower NaCl concentrations may widen the safe density window of the drilling fluid in the hydrate formation under the same conditions. The fluid solute concentration increases from 0.05 wt % to 0.2 wt %, and the safe density window decreases from a range of 1.38 g/cm³-1.47 g/cm³ to a range of 1.40 g/cm³-1.45 g/cm³ after 6 hours. The analytical process may also be extended to the remaining drilling fluid treatments and solutions.

Embodiments of the present disclosure provide the method for calculating the safe density window of the hydrate formation considering a mud cake. The method includes establishing the seepage model of the mud cake and the seepage model of the hydrate formation, solving the saturation distribution of formation water, the saturation distribution of methane gas, and the saturation distribution of hydrate based on the finite element software, determining, based on the mass conservation equation of hydrate-bearing formation solute and the mass conservation equation of mud cake solute, the solute transport model of the mud cake and a solute transport model of the hydrate formation, and solving the solute solubility distribution based on the finite element software.

The method also includes constructing the heat transfer model of the mud cake and the heat transfer model of the hydrate formation based on the heat transfer equation of the hydrate formation and the heat transfer equation of the mud cake and solving the temperature distribution of a well wall based on the finite element software.

The method also includes constructing the skeletal mechanical model of the mud cake and the skeletal mechanical model of the hydrate formation, solving rock deformation around the well based on the finite element software, coupling and solving the pore pressure distribution at the well wall based on the energy field equation, and deriving the effective stress distribution at the well wall by using the programming software according to the pore pressure distribution, based on the pore pressure distribution at the well wall.

The method further includes determining the collapse pressure and the rupture pressure based on the solved effective stress distribution of the well wall according to the Cullen-Moore criterion and the tensile damage criterion, to establish the manner for calculating the safe density window of the hydrate formation under the action of the drilling fluid.

More descriptions regarding the description may be found in the corresponding description hereinabove.

The present disclosure also discloses a computer device, which includes a memory and a processor. The memory stores a computer program, and the processor implements the foregoing method when executing the computer program.

The present disclosure further discloses a non-transitory computer-readable storage medium storing a computer program. When a processor executes the computer program, the processor implements the foregoing method.

### Embodiment 3

The embodiment 3 of the present disclosure has the following differences from the first two embodiments.

The described functionality may be stored in the non-transitory computer-readable storage medium if it is implemented in the form of a software functional unit and sold or used as a separate product. Based on this understanding, the technical solution of the present disclosure, the part that contributes to the prior art, or the part of the technical solution is embodied in the form of a computer software product. The computer software product is stored in a storage medium and includes a plurality of instructions to cause a computer device (which may be a personal computer, a server, or a network device, etc.) to perform all or some of the operations of the method described in various embodiments of the present disclosure. The storage media include USB flash drives, mobile hard disks, read-only memory (ROM), random access memory (RAM), magnetic disks, or compact disc, and other kinds of media that can store program code.

The present disclosure is described with reference to flowcharts and/or block diagrams of methods, apparatus (systems), and computer program products according to embodiments of the present disclosure. It is to be understood that a combination of each of the processes and/or boxes in the flowchart and/or block diagram, and the processes and/or boxes in the flowchart and/or block diagram, may be implemented by the computer program instructions. These computer program instructions may be provided to a processor of a general-purpose computer, a special-purpose computer, an embedded processor, or other programmable data-processing device to produce a machine such that the instructions executed by the computer or other programmable data processing device's processor produce a device for implementing a function specified in one or more processes of a flowchart and/or one or more boxes of a block diagram.

These computer program instructions may also be stored in a computer-readable memory capable of directing a computer or other programmable data-processing device to operate in a particular manner, such that the instructions stored in that computer-readable memory produce a manufacturer including an instruction device. The instruction device implements a function specified in one or more processes of the flowchart and/or one or more boxes of the block diagram.

These computer program instructions may also be loaded onto a computer or other programmable data processing device such that a series of operations are performed on the computer or other programmable device to produce a computer-implemented process. Thus, the instructions executed on the computer or other programmable device provide operations for implementing a function specified in one or more processes of the flowchart and/or one or more boxes of the block diagram.

What is claimed is:

1. A method for determining a safe density window of a hydrate formation considering a mud cake, the method being performed by a processor, comprising,
establishing a seepage model of the mud cake and the hydrate formation, determining a saturation distribution of formation water, a saturation distribution of methane gas, and a saturation distribution of hydrate based on a finite element software, determining, based on a mass conservation equation of hydrate-bearing formation solute and a mass conservation equation of mud cake solute, a solute transport model of the mud cake and a solute transport model of the hydrate formation, and determining a solute solubility distribution based on the finite element software;
constructing a heat transfer model of the mud cake and a heat transfer model of the hydrate formation based on a heat transfer equation of the hydrate formation and a heat transfer equation of the mud cake, and determining a temperature distribution of a well wall based on the finite element software;
constructing a skeletal mechanical model of the mud cake and a skeletal mechanical model of the hydrate formation, determining rock deformation around a well based on the finite element software, coupling and determining a pore pressure distribution at the well wall based on an energy field equation, and deriving an effective stress distribution at the well wall by using a programming software based on the pore pressure distribution at the well wall;
determining a collapse pressure and a rupture pressure based on the effective stress distribution according to a Cullen-Moore criterion and a tensile damage criterion, to obtain the safe density window of an interaction between a drilling fluid and the hydrate formation;
collecting a drilling fluid density by density measuring equipment;
determining, in response to the drilling fluid density not meeting a preset condition, a density adjustment amount, wherein the preset condition is set based on the safe density window; and
controlling an operation of a density adjustment apparatus based on the density adjustment amount.

2. The method of claim 1, further comprising:
selecting, in response to the drilling fluid density not meeting the preset condition, a target drilling fluid density from the safe density window, including:
determining, in response to the drilling fluid density not meeting the preset condition, the target drilling fluid density based on an upper limit and a lower limit of the safe density window.

3. The method of claim 1, further comprising:
collecting underground environmental data through monitoring equipment.
determining a correction window by a correction model based on the safe density window, pressure data and density data of the drilling fluid, a chemical composition concentration of the drilling fluid, and the underground environmental data, the correction model being a machine learning model; and
correcting the safe density window based on the correction window.

4. The method of claim 3, further comprising:
updating initial conditions of the seepage model, the heat transfer model, and the skeletal mechanical model based on the underground environmental data.

5. The method of claim 1, further comprising:
generating a plurality of sets of candidate adjustment parameters based on the drilling fluid density and a target drilling fluid density by a parameter determination model, the parameter determination model being a machine learning model; and
determining an optimal adjustment parameter based on the plurality of sets of candidate adjustment parameters.

6. The method of claim 1, wherein the establishing a seepage model of the mud cake and the hydrate formation includes obtaining, from a continuity equation and a generalized Darcy's law, seepage information, wherein
a change rate of a mass of the methane gas in the hydrate formation over time is equal to a flow rate of permeability of the hydrate formation under an action of a pressure gradient of the methane gas plus a gas production rate of hydrate dissociation;
a change rate of a mass of water in the hydrate formation over time is equal to a flow rate of permeability of formation under an action of a pressure gradient of the water plus a water production rate of the hydrate dissociation; and
a change rate of a mass of the hydrate in the hydrate formation over time is equal to a hydrate rate of the hydrate dissociation; and
assuming that only an aqueous phase exists in pores of the mud cake and that a flow is in accordance with the generalized Darcy's law, then
a product of a change rate of a pressure of the formation water in the mud cake over time and a ratio of a porosity of the mud cake to a shear modulus of the mud cake is equal to a flow rate of the formation water in the mud cake.

7. The method of claim 6, wherein the determining a saturation distribution of formation water, a saturation distribution of methane gas, and a saturation distribution of hydrate includes determining a decomposition rate of the hydrate by:
determining the decomposition rate of the hydrate by a ratio of a contact area of the hydrate contacting a water interface to an Avogadro constant, a ratio of water molecules to gas molecules, a collision cross-sectional area of the water, a dissolution kinetic constant, a collision cross-sectional area of gas, and a desorption kinetic constant, wherein
the desorption kinetic constant is determined by a self-diffusion coefficient of the gas, a proportion of an uncovered surface of the hydrate, gas composition, and a relationship between a fugacity of the gas molecules and an equilibrium fugacity in a liquid phase; and
the dissolution kinetic constant is determined by a self-diffusion coefficient of the water molecules, water molecule composition and enthalpies of phase transitions of hydrate lattice, a blocking coefficient of solute diffusion, a length of a core sample, a temperature, an initial temperature, and a gas constant;
a relationship between an output rate of the methane gas and the water and the decomposition rate of the hydrate being:
the output rate of the methane gas being proportional to the decomposition rate of the hydrate, with a proportionality coefficient being a ratio of a mass fraction of the methane gas to a mass fraction of the hydrate and sign being opposite; and
the output rate of the water being proportional to the decomposition rate of the hydrate, with a proportionality coefficient being a ratio of a mass fraction of the water to the mass fraction of the hydrate and sign being opposite;
a relationship between an absolute permeability of the hydrate formation and a saturation of the hydrate being:
the absolute permeability of the hydrate formation being determined by a relationship between an intrinsic permeability of a hydrate-free sediment and the saturation of the hydrate, wherein a permeability decline index characterizes an extent to which the permeability varies with the hydrate saturation; and
a linear relationship between a mechanical strength parameter of a layer containing the hydrate and a saturation degree of the layer containing the hydrate being:
a cohesion of the hydrate formation being determined by a cohesion at the saturation of the hydrate of 0 and a linear relationship between a perturbed cohesion of the hydrate and the saturation of the hydrate, wherein the perturbed cohesion of the hydrate is a constant.

8. The method of claim 7, wherein the determining a solute transport model of the mud cake and a solute transport model of the hydrate formation includes a relationship of the mass conservation equation of the hydrate-bearing formation solute, including:
the change rate of solute concentration in the hydrate formation over time being balanced with diffusion amount of solute in the hydrate formation; and
a solute diffusion coefficient in the hydrate formation being determined by a water saturation in the hydrate formation and a solute diffusion coefficient of the hydrate-free sediment; and
a relationship of the mass conservation equation of the mud cake solute, including:
a change rate of solute concentration over time being balanced with a diffusion amount of solute in the mud cake; and
a solute diffusion coefficient in the mud cake being determined by the porosity of the mud cake, a porosity of the hydrate formation, and a solute diffusion coefficient of the hydrate-free sediment.

9. The method of claim 8, wherein the constructing a heat transfer model of the mud cake and a heat transfer model of the hydrate formation includes a heat transfer relationship in a water-bearing sediment controlled by heat conduction and heat convection, including:
a heat exchange in the water-bearing sediment being affected by both heat conduction and heat convection, and the heat exchange in the water-bearing sediment being balanced with heat released during a hydrate decomposition process;
an equivalent specific heat of a sediment being determined by a heat capacity of a skeleton of the hydrate formation and a heat capacity of the hydrate, the water, and the methane gas; and
a thermal conductivity of the water-bearing sediment being determined by a thermal conductivity of the hydrate formation and a thermal conductivity of the water, the methane gas, and the hydrate; and
a heat transfer relationship in the mud cake, including:
a variation of a heat energy of the mud cake over time being balanced with an amount of heat conduction.

10. The method of claim 9, wherein the constructing a heat transfer model of the mud cake and a heat transfer model of hydrate formation further includes obtaining a matrix equilibrium relationship of the hydrate formation based on an effective stress principle and an elastic-plastic mechanics theory, including:
balancing an effective stress gradient of the hydrate formation with a gradient of a product of a pore pressure and a Biot constant;
setting a value of a Kronecker preset function to 1 when an action direction of an effective stress in the hydrate formation is the same as a plane direction of the action direction applied to the hydrate formation; and
setting a value of the Kronecker preset function to 0 when the action direction of the effective stress in the hydrate formation is different from the plane direction of the action direction applied to the hydrate formation.

11. The method of claim 10, wherein the constructing a heat transfer model of the mud cake and a heat transfer model of hydrate formation further includes a tensor form of geometric equations, indicating that:
the strain tensor is determined by an average value of a total displacement u_(i, j) and a total displacement u_(j, i), wherein u denotes the total displacement, i denotes the action direction of the effective stress of the hydrate formation, and j denotes the plane direction of the action direction applied to the hydrate formation;
based on elastic-plastic constitutive equations and a Drucker-Prager yield criterion, an incremental form of an elastic constitutive equation indicating that an effective stress increment is determined by an elastic-plastic matrix tensor and a strain increment; and
for straight wells, non-uniform horizontal in situ ground stresses, and taking into account fluid percolation and the pore pressure, a stress state in a well wall envelope being that:
a radial stress of the well wall envelope is determined by a product of a well wall pressure and the pore pressure with the Biot constant;
a tangential stress of the well wall envelope is determined by a maximum horizontal principal stress, a minimum horizontal principal stress, the wellbore pressure, the pore pressure, and an angle; and
a pendent stress of the well wall envelope is determined by a vertical stress, the maximum horizontal principal stress, the minimum horizontal principal stress, the well wall pressure, the pore pressure, and the angle.

12. The method of claim 11, wherein under an action of the drilling fluids, and based on a Mohr-Coulomb criterion, the method comprises:
when σᵣ serving as a minimum principal stress and σ_{θ} serving as a maximum principal stress,
determining the minimum principal stress σᵣ by the product of the wellbore pressure and the pore pressure with the Biot constant; determining the maximum principal stress by a product of the maximum horizontal principal stress, the minimum horizontal principal stress, the wellbore pressure and the pore pressure with the Biot constant;
according to the Mohr-Coulomb criterion, when σᵣ serving as the minimum principal stress and σ_{θ} serving as the maximum principal stress,
determining the collapse pressure by the maximum principal stress, the minimum principal stress, an internal friction angle, and a cohesion;
when σᵣ serving as the minimum principal stress and σ_{z} serving as the maximum principal stress,
determining the minimum principal stress σᵣ by the product of the wellbore pressure and the pore pressure with the Biot constant; determining the maximum principal stress σᵣ by the vertical stress, a Poisson ratio, the maximum horizontal principal stress, the minimum horizontal principal stress, and the product of the pore pressure and the Biot constant;
according to the Mohr-Coulomb criterion, when σᵣ serving as the minimum principal stress and σ_{z} serving as the maximum principal stress, determining the collapse pressure by the maximum principal stress, the minimum principal stress, the internal friction angle, and the cohesion;
when σ_{θ} serving as the minimum principal stress and σ_{z} serving as the maximum principal stress,
determining the minimum principal stress σ_{θ} by the product of the maximum horizontal principal stress, the minimum horizontal principal stress, the wellbore pressure, and the pore pressure with the Biot constant; and determining the maximum principal stress σ_{z} by a product of the vertical stress, the Poisson ratio, the maximum horizontal principal stress, the minimum horizontal principal stress, and the pore pressure with the Biot constant;
according to the Mohr-Coulomb criterion, when σ_{θ} serving as the minimum principal stress and σ_{z} serving as the maximum principal stress, determining the collapse pressure by the maximum principal stress, the minimum principal stress, the internal friction angle, and the cohesion;
when σ_{θ} serving as the minimum principal stress and σr serving as the maximum principal stress,
determining the minimum principal stress σ_{θ} by the product of the maximum horizontal principal stress, the minimum horizontal principal stress, the wellbore pressure, and the pore pressure, with the Biot constant; and determining the maximum principal stress σᵣ by the product of the wellbore pressure and the pore pressure with the Biot constant;
according to the Mohr-Coulomb criterion, when σ_{θ} serving as the minimum principal stress and σᵣ serving as the maximum principal stress, determining the collapse pressure by the maximum principal stress, the minimum principal stress, the internal friction angle, and the cohesion;
when tensile damage occurring in formation,
determining the collapse pressure by the product of the minimum horizontal principal stress, the maximum horizontal principal stress, and the pore pressure with the Biot constant, the tensile strength, and the wellbore pressure; and
a manner for determining a density window of safe drilling fluid including:
determining the density window of the safe drilling fluid by the collapse pressure when the tensile damage occurs in the formation and a minimum value of the collapse pressure under different conditions.

13. A computer device, comprising a memory and a processor, wherein the memory stores a computer program, and the processor implements the method of claim 1 when executing the computer program.

14. A non-transitory computer-readable storage medium storing a computer program, wherein when a processor executes the computer program, the processor implements the method of claim 1.
